(19) United States
(12) Patent Application Publication
(10) Pub. No.: US 2021/0192734 A1
(43) Pub. Date: Jun. 24, 2021

(54) ELECTRONIC METHOD AND DEVICE FOR AIDING THE DETERMINATION, IN AN IMAGE OF A SAMPLE, OF AT LEAST ONE ELEMENT OF INTEREST FROM AMONG BIOLOGICAL ELEMENTS, ASSOCIATED COMPUTER PROGRAM

(71) Applicant: KEEN EYE TECHNOLOGIES, Paris (FR)

(72) Inventors: Cécile MALÉCOT, ASNIERES (FR); Eric PELTIER, CLAMART (FR)

(21) Appl. No.: 16/092,331

(22) PCT Filed: Apr. 4, 2017
(86) PCT No.: PCT/EP2017/058019

§ 371 Date: Oct. 9, 2018

(30) Foreign Application Priority Data
Apr. 6, 2016 (FR) 1653017

(51) Int. Cl. G06T 7/00; G06T 7/11; G16H 30/20

(52) U.S. Cl. CPC G06T 7/0014; G06T 2207/30024; G16H 30/20; G06T 7/11

(57) ABSTRACT

Disclosed is a method for assisting the determination, in an image of a sample, of at least one element of interest among a plurality of biological elements, the sample including the plurality of biological elements, is implemented by an electronic device. This determination assistance method includes the acquisition of an image of the sample; the preselection of at least one zone in the acquired image, from a database including a set of reference element thumbnails; the display of each zone preselection in the image; the acquisition of the selection by an operator of at least one zone among the preselected zones, each selected zone corresponding to at least one element of interest; and the updating of the database according to the selected zone or zones acquired, at least one selected zone acquired being added as a reference element thumbnail in the base.

The present invention relates to a method for assisting the determination, in an image of a sample, of at least one element of interest among a plurality of biological elements, the sample comprising the plurality of biological elements, and the method being implemented by an electronic device.

The invention also relates to a computer program containing software instructions which, when they are executed by a computer, implement such a method.

The invention also relates to an electronic device for assisting the determination, in an image of a sample, of at least one element of interest among biological elements contained in the sample.

The analysis of an image of a biological sample, such as a sample of cells corresponding either to a tissue or to a cell suspension, living or fixed, is an extremely tricky operation. The diagnosis of this biological sample, in particular cytological and histological diagnosis, covers in fact the diagnosis techniques based in particular on the morphological examination of the cells. It is very well suited to the screening of cancer and precancerous lesions, in particular on the neck of the uterus.

Analysis of the image of the biological sample is generally carried out by a cytotechnician or a technician initially, or more frequently by a pathologist or a doctor secondly, in particular in the case of doubt as to the nature of certain biological elements contained in the sample, the aim being to screen pathological elements, such as pathological cells.

The first operator, such as for example a technician, a cytotechnician, a pathologist or a doctor, generally examines a plurality of images of various samples in succession, and then next transmits to a second operator, for example a pathologist or a doctor, the images requiring a more detailed analysis, as well as any other images, expected by the pathologist or doctor.

However, such an analysis process may prove to be relatively lengthy and tedious, and is therefore not optimum. Furthermore, it sometimes happens that an error in diagnosis occurs when the biological sample is particularly difficult to interpret.

The aim of the invention is then to overcome these drawbacks by proposing a method for assisting the determination of the element or elements of interest in the sample image, the method being implemented by an electronic device.

To this end, the subject matter of the invention is a method for assisting the determination, in an image of a sample, of at least one element of interest among a plurality of biological elements, the sample comprising the plurality of biological elements,

the method being implemented by an electronic device, and comprising:
acquisition of an image of the sample;
preselection of at least one zone in the acquired image, from a database comprising a set of thumbnails of reference elements;
display of each zone preselection in the image;
acquisition of the selection by an operator of at least one zone among the preselected zones, each selected zone corresponding to at least one element of interest; and
updating of the database according to the selected zone or zones acquired, at least one selected zone acquired being added as a thumbnail of a reference element in said base.

The determination assistance method according to the invention then preselects at least one zone in the acquired image, from a database comprising a set of reference element thumbnails, in order to facilitate analysis of the sample.

Furthermore, the determination assistance method then takes into account the feedback from the operator, by acquiring the selection of zone or zones made by the operator among the preselected zones, each selected zone corresponding to at least one element of interest, and updating the database accordingly, in order to improve the relevance of the next preselections that will be made by the electronic device.

According to other advantageous aspects of the invention, the determination assistance method comprises one or more of the following features, taken in isolation or in accordance with all technically possible combinations:
the preselection of at least one zone in the acquired image comprises:
   +the detection of a plurality of zones in the image, each zone corresponding to at least one biological element, and
   +the comparison of each detected zone with the set of reference element thumbnails, the zone or zones being preselected according to said comparison;
during the comparison, a value of at least one parameter relating to a corresponding biological element is calculated for each zone detected, and each calculated parameter value is compared with the corresponding parameter values of all the reference element thumbnails;
each parameter is chosen from the group consisting of: a shape, a diameter, a surface, a volume, a perimeter, a mass, a colour and a texture;
each biological element is chosen from the group consisting of: a cytoplasm, an organite, a vesicle, a membrane, a nucleus, a cell, an intracellular pathogen, an extracellular pathogen, a unicellular organism, a pluricellular organism, a bacterium, a virus, a mycelium, a fragment of organism, an organism, an organ fragment, an organ, a tissue, a biological structure fragment, a biological structure, an anatomical structure fragment and an anatomical structure;
during the preselection, a plurality of zones are preselected and, during the display, the zone preselections are displayed successively;
during the display, the zone preselections are displayed in an order dependent on a degree of agreement of each preselection;
the degree of agreement of each preselection is calculated according to a difference between the preselected zone and the reference element thumbnail associated during the comparison;
the database comprises a relevance index for each thumbnail of a reference element and, during the preselection, the relevance index is increased for each reference element thumbnail associated with a preselected zone;
during the preselection, the reference element thumbnails are taken into account in an order dependent on their respective relevance index; and
each biological element is chosen from the group consisting of: a cytoplasm, an organite, a vesicle, a membrane, a nucleus, a cell, an intracellular pathogen, an extracellular pathogen, a unicellular organism, a pluricellular organism, a bacterium, a virus, a mycelium, a fragment of organism, an organism, an organ fragment, an organ, a tissue, a biological structure fragment, a biological structure, an anatomical structure fragment and an anatomical structure.

Another subject matter of the invention is a computer program containing software instructions which, when they are executed by a computer, implement the method as defined above.

Another subject matter of the invention is an electronic device assisting the determination, in an image of a sample, of at least one element of interest among a plurality of biological elements, the sample comprising the plurality of biological elements,

the electronic device comprising:
a first acquisition module configured to acquire an image of the sample;
a preselection module configured to preselect at least zone in the acquired image, from a database comprising a set of reference element thumbnails;
a display module configured to display each zone preselection in the image;
a second acquisition module configured to acquire the selection by an operator of at least one zone among the preselected zones, each selected zone corresponding to at least one element of interest; and
an updating module configured to update the database according to the selected zone acquired or zones, the updating module being configured to add, as a thumbnail of a reference element in said base, at least one selected zone acquired.

These features and advantages of the invention will emerge from a reading of the following description given solely by way of non-limitative example and made with reference to the accompanying drawings, on which:

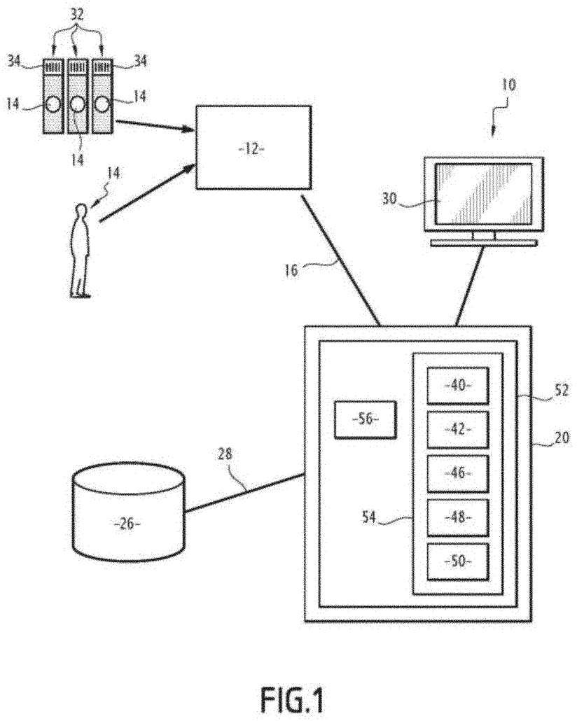

FIG. 1 is a schematic representation of a device for reading a living sample (radiological images) or fixed sample (anatomy and pathological cytology images) that has been digitised and a system for assisting the determination, in an image of a sample, of at least one element of interest among a plurality of biological elements, the determination assistance system being connected to the reading device and comprising an electronic determination-assistance device according to the invention, a display screen and a database; and

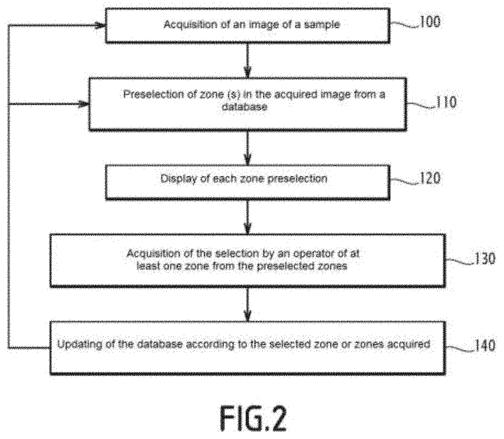

FIG. 2 is a flow diagram of a determination assistance method according to the invention.

In FIG. 1, a determination assistance system 10 is connected to a device 12 for reading a sample 14 by means of a first data link 16.

The determination assistance system 10 comprises an electronic device 20 for assisting the determination, in an image of the sample 14, of at least one element of interest among a plurality of biological elements, the sample 14 comprising the plurality of biological elements.

The determination assistance system 10 comprises a database 26, connected to the electronic determination-assistance device 20 by means of a second data link 28. In a variant that is not shown, the database 26 is integrated in the electronic determination-assistance device 20.

The determination assistance system 10 comprises a display screen 30 connected to the electronic determination-assistance device 20.

The determination assistance system 10, in particular the electronic determination-assistance device 20, is configured to assist in determining, in an image of the sample 14, at least one element of interest among a plurality of biological elements.

“Element of interest” means a biological element liable to interest the operator, such as the technician or the cytotechnician of the pathologist or the doctor, such an element of interest being in particular a pathological element, such as an abnormal structure or cell, in particular when the determination assistance system 10 is used to facilitate the diagnosis of at least one pathology associated with the sample 14.

The reading device 12 is known per se, and is configured to read the sample 14, that is to say to obtain an image of the sample 14. The reading device 12 is also referred to as a digitisation device, the image obtained being a digital image.

The sample 14 is for example disposed on a plate 32, the plate 32 being able to be inserted in the reading device 12, and then read by the latter. Each plate 32 comprises a unique identifier 34, for example in the form of a bar code, to uniquely identify the sample 14 associated with the plate 32. In a variant or in addition, the sample 14 means more broadly a human being, for example in the case where the reading device 12 is in particular a medical imaging device, or a radiology device.

The first data link 16 is for example a cabled connection. In a variant, the first data link 16 is a radio connection.

The electronic determination-assistance device 20 comprises a first acquisition module 40 configured to acquire an image of the sample 14, and a preselection module 42 configured to preselect at least one zone in the image acquired from the database 26.

The electronic determination-assistance device 20 comprises a display module 46 configured to display, for example on the display screen 30, each preselection of a zone in the image, and a second acquisition module 48 configured to acquire the selection by an operator, not shown, of at least one zone among the preselected zones, each selected zone corresponding to at least one element of interest.

The electronic determination-assistance device 20 comprises an updating module 50 configured to update the database 26 according to the selected zone or zones acquired, the updating module 50 being configured to add, as a thumbnail of a reference element in said database, each selected zone acquired.

In the example in FIG. 1, the electronic determination-assistance device 20 comprises an information processing unit 52 formed for example by a memory 54 and a processor 56 associated with the memory 54.

Each biological element is chosen from the group consisting of: a cytoplasm, an organite, a vesicle, a membrane, a nucleus, a cell, an intracellular pathogen, an extracellular pathogen, a unicellular organism, a pluricellular organism, a bacterium, a virus, a mycelium, a fragment of organism, an organism, an organ fragment, an organ, a tissue, a biological structure fragment, a biological structure, an anatomical structure fragment and an anatomical structure. For example, the biological elements contained in the sample 14 are biological cells or a cluster of biological cells.

The database 26 comprises a set of reference element thumbnails, this set being used to preselect at least one zone in the acquired image.

In an optional supplement, the database 26 comprises a relevance index for each thumbnail of a reference element. The relevance index is an indicator quantifying the probability that a thumbnail of a reference element is used for the preselection of at least one zone in the acquired image. The higher this probability, the higher the relevance index. In other words, the more a thumbnail is representative of an element of interest, the higher its relevance index.

In the example in FIG. 1, the database 26 is a local database connected to the electronic determination-assistance device 20 by means of the second data link 28, the second data link 28 being for example a cabled connection. In a variant, the second data link 28 is a radio connection.

In a variant that is not shown, the database 26 is a remote database connected to the electronic determination-assistance device 20 via a communication network. The second data link 28 then corresponds to the communication network, such as the internet.

In the example in FIG. 1, the first acquisition module 40, the preselection module 42, the display module 46, the second acquisition module 48 and the updating module 50 are each implemented in the form of software. The memory 54 of the information processing unit 52 is then able to store first acquisition software configured to acquire an image of the sample, preselection software configured to preselect at least one zone in the image acquired from the database 26, display software configured to display each zone preselection, second acquisition software configured to acquire the selection by the operator of at least one zone among the preselected zones, and updating software configured to update the database 26 according to the selected zone or zones acquired, the updating software being configured to add, as a thumbnail of a reference element in said base 26, each selected zone acquired. The processor 56 of the information processing unit 52 is then able to execute the first acquisition software, the preselection software, the display software, the second acquisition software and the updating software.

In a variant that is not shown, the first acquisition module 40, the preselection module 42, the display module 46, the second acquisition module 48 and the updating module 50 are each implemented in the form of a programmable logic component, such as an FPGA (field-programmable gate array) or in the form of a dedicated integrated circuit such as an ASIC (application-specific integrated circuit).

The first acquisition module 40 is configured to acquire, that is to say to obtain, each image of the sample 14. The first acquisition module 40 is for example configured to obtain the image from the reading device 12 in the example in FIG. 1.

The preselection module 42 is configured to preselect at least one zone in the image acquired from the database 26, for example by detecting a plurality of zones in the image, each zone corresponding to at least one biological element, and then by comparing each detected zone with the set of reference element thumbnails, contained in the database 26, the zone or zones then being preselected according to the result of this comparison.

In general terms, each preselected zone is a zone of the image representing one or more biological elements, this preferably being centred on the biological element when only one biological element is visible in said zone.

For the zone detection, the preselection module 42 is for example configured to use known imaging processing algorithms, such as algorithms of the mathematical morphological type, with as a basic operator at least one operator chosen from the group consisting of: a closing operator, an opening operator, an erosion operator, an expansion operator, a transformation operator and a thresholding operator. A segmentation of the image to distinguish and separate the zones from each other is carried out by the preselection module 42 by applying one or more known techniques from the following techniques, the non-exhaustive list of which proposed here by way of example comprises: watershed, contour recognition (gradient, Laplacian), thresholding.

For the comparison of each zone detected with all the reference element thumbnails, the preselection module 42 is configured to calculate, for each zone detected, a value of at least one parameter relating to a corresponding biological element. The preselection module 42 is also configured to compare each parameter value calculated with corresponding parameter values of the set of reference element thumbnails.

Each biological element is, for example, chosen from the group consisting of: a cytoplasm, an organite, a vesicle, a membrane, a nucleus, a cell, an intracellular pathogen, an extracellular pathogen, a unicellular organism, a pluricellular organism, a bacterium, a virus, a mycelium, a fragment of organism, an organism, an organ fragment, an organ, a tissue, a biological structure fragment, a biological structure, an anatomical structure fragment and an anatomical structure.

Each parameter is then chosen for example from the group consisting of: a shape, a diameter, a surface, a volume, a perimeter, a mass, a colour and a texture.

The diameter, or the size, of the element in question is for example determined by counting pixels in a given direction.

The surface area of the element in question is for example determined by counting the number of pixels corresponding to said surface area.

The perimeter of the element in question is for example determined by counting the number of pixels forming the periphery of said element.

The shape of the element corresponds for example to the skeleton obtained by mathematical morphology. When the element is a cell, the shape is for example a disc for a superficial or intermediate cell, and a disc or a polygon for a parabasal or basal cell. When the element is a normal nucleus, the shape is for example a disc, and when the element is a pathological nucleus, the shape is generally a polygon and, in some cases, a disc. The shape of the nucleus is then a good indicator for detecting pathology.

The volume and/or the mass of the element in question are estimated from a counting of the pixels carried out on the image 22 in two dimensions.

The volume of the element in question is for example estimated by determining a dimension, or size, of the element in question in a given direction with the counting of the number of pixels in this direction, and then by extrapolation of the volume from the dimension determined. By way of example, when the element in question is in the form of a sphere, its diameter, or its radius, in the image 22 is determined by counting the number of pixels, and the volume of the sphere is next calculated from this estimated diameter or radius.

The mass of the element in question is estimated from an estimated value of its volume, as well as a colour or a texture of the element in question. A dark colour or texture with identifiable patterns indicates for example a greater density of matter, and therefore a higher mass.

The colour of the element in question corresponds to a colour of the pixels forming the element in question, in particular to an average of the colour values of these pixels forming the element in question, expressed as a grey level or colour level, for example using an RGB colour model.

The texture of the element in question corresponds to a characteristic pattern, regular or random, recognisable by various methods known per se, such as fractal analysis, statistical analysis or a Markov field approach.

A person skilled in the art will of course understand that the comparison of the zone detected with the thumbnails contained in the database 26 is preferably carried out for the same element and the same parameter, in order to make the comparison on comparable data.

A person skilled in the art will also understand that the type of element and/or the type of parameter is liable to vary from one comparison to another.

As an optional addition, the comparison of the detected zone with the thumbnails contained in the database 26 is carried out for a predefined set of elements and a predefined set of parameters, each comparison then not necessarily being carried out for the same elements and the same parameter, in order better to distinguish the comparisons made with the same element and the same parameter.

As an optional addition, the preselection module 42 is further configured to take into account the thumbnails contained in the database 26 in an order dependent on their respective relevance index. This makes it possible to carry out the preselection of zones more quickly.

Furthermore, the preselection module 42 is configured to increase the relevance index for each thumbnail associated with a preselected zone. In other words, whenever a zone is preselected by comparison with a given reference element thumbnail, the relevance index of said thumbnail is increased by the preselection module 42.

The preselection module 42 is then configured to preselect one or more zones in the image, and, in the event of multiple preselection, the various zone preselections are displayed successively by the display module 46, as will be described hereinafter.

The display module 46 is configured to display each zone preselection in the image, in particular to display the preselected zone differently compared with any other zones detected.

As an optional addition, when a plurality of zones are preselected by the preselection module 42, the display module 46 is configured to display the preselection zones successively.

As an optional addition, the display module 46 is further configured to display the zone preselections in an order dependent on a degree of agreement of each preselection. The degree of agreement of each preselection is calculated according to a difference between the preselected zone and the reference element thumbnail that was taken into account during the comparison.

The second acquisition module 48 is configured to acquire the selection of at least one zone, carried out by an operator such as a technician or a cytotechnician or a pathologist or a doctor, among the zone or zones previously preselected by the preselection module 42. The selection made by the operator is carried out using an entry device, not shown and known per se, such as for example a keyboard, a mouse or a touchscreen. The second acquisition module 48 is then configured to acquire, that is to say to obtain, from the entry device, the selection made by the operator. Each selected zone corresponds to at least one element of interest for the operator.

The updating module 50 is configured to update the database 26 from the zone or zones acquired by the second acquisition module 48, at least one selected zone acquired being added in the database 26 as thumbnails of a reference element. In other words, the updating module 50 makes it possible to automatically update the database 26 when the operator has selected a new zone, this selection meaning that the zone corresponds to a biological element of interest. This then makes it possible to enrich the database 26 with a thumbnail corresponding to this new biological element of interest.

In addition, the updating module 50 is configured also to store in the database 26 the parameter or parameters calculated for each thumbnail added.

As an optional addition, the updating module 50 is configured to update the database 26 only in the case of the reception also of a validation indication, this validation indication resulting from the validation by another operator, for example a pathologist or a doctor, of the change to the database 26, in particular of the addition in the database 26 of the thumbnail corresponding to the selected zone acquired. In a variant, the validation of the modification of the database 26 in order to obtain the validation indication is done by the same operator, and is in this case different over time, with a review and a validation of the changes to be made to the database 26.

According to this optional addition, the updating module 50 is also configured to eliminate the selected zone acquired in the case of the reception of a deletion indication, this deletion indication resulting for example from the rejection by the other operator, or the same operator, of the change to the database 26.

The functioning of the electronic determination assistance device 20 will now be described with the help of FIG. 2 illustrating a flow diagram of the determination assistance method according to the invention.

During an initial step 100, the first acquisition module 40 acquires a respective image of the sample 14, for example from the reading device 12.

The preselection module 42 next preselects, during the following step 110, one or more zones in the image acquired during the previous step, from the database 26. The preselection is for example made by detecting a plurality of zones in the image, each zone corresponding to at least one biological element, and then comparing each zone detected with the thumbnails contained in the database 26, the zone or zones being preselected according to the result of said comparison.

As indicated previously the zone detection is for example made by using known image processing algorithms, in particular of the mathematical morphology type. The comparison of each zone detected with the thumbnails contained in the database 26 next comprises the calculation, for each zone detected, of a value of at least one parameter relating to an element of a corresponding biological element, and the comparison, for each parameter, of the value calculated for the zone detected with the value for the thumbnail contained in the database 26.

In addition, a plurality of values of the same parameter are calculated, and the comparison is then made using an average of these values, or using a tolerance spread around a median value.

As an optional addition, the database 26 comprises a relevance indicator for each thumbnail that it contains and, during the preselection step 110, the relevance index is increased for each reference element thumbnail associated with a preselected zone.

According to this optional addition, during the preselection step 110, the reference element thumbnails are taken into account preferably in an order dependent on their respective relevance index, the most relevant thumbnails being taken into account first, or as a priority.

During the following step 120, the display module 46 displays each zone preselection, the preselected zone then being for example displayed differently compared with a simply detected zone.

When a plurality of zones are preselected during step 110, the zone preselections are then displayed successively during step 120. As an optional addition, the zone preselections are then displayed in an order dependent on the degree of agreement of each preselection, the preselection with the greatest agreement being displayed first, or as a priority.

The second acquisition module 48 next acquires, during the following step 130, each selection, made by the operator, of at least one zone among the zones preselected during step 110, displayed during step 120.

During the following step 140, the updating module 50 determines then whether it is necessary to update the database 26 according to the selected zone or zones acquired.

Each selected zone acquired is for example added as a thumbnail of a reference element in the database 26.

In a variant, the database 26 is updated with the addition of a selected zone acquired only if the corresponding validation indication is received by the updating module 50, this validation indication being generated on agreement by the other operator, or the same operator, for this modification of the database 26, in particular for the addition in the database 26 of said thumbnail corresponding to the selected zone acquired.

According to this variant, if the other operator, or the same operator, does not validate this change, but on the contrary rejects it, this gives rise to the generation of the deletion indication. When the updating module 50 receives this deletion indication, it then deletes the selected zone acquired, and the database 26 is therefore not updated in this case.

At the end of step 140, the determination assistance method returns to step 100 in order to acquire a new image of the sample 14, or to step 110 in order once again, by means of the updated database 26, to make a preselection of a zone or zones in the previously acquired image.

Thus the electronic determination assistance device 20 and the determination assistance method according to the invention make it possible to select at least one zone in the acquired image, from the database 26 comprising the reference element thumbnails, and then to display this preselection intended for the operator, in order to facilitate analysis of the sample 14 by the operator.

This is because the electronic determination assistance device 20 then puts forward in the acquired image, that is to say reveals, the zone or zones that are the most liable to contain an element of interest.

Furthermore, the electronic determination assistance device 20 and the associated method according to the invention next make it possible to take into account the feedback of the operator, by acquiring the selection of a zone or zones made by the operator among the preselected zones, each selected zone corresponding to at least one element of interest, and updating the database accordingly.

This therefore improves the relevance of the next preselections that will be made by the electronic determination assistance device 20. In other words, this updating of the database 26 and then the looping back with the use of the database 26 updated for the next selection, then allows self-learning by the electronic determination assistance device 20.

It will thus be understood that the electronic determination assistance device 20 and the associated method according to the invention make it possible to accelerate the process of analysis of the sample 14 while limiting the risks of diagnostic error, by providing an assistance to the determination of the element or elements of interest visible in the image of the sample 14.

1-13. (canceled)

14. Method for assisting the determination, in an image of a sample, of at least one element of interest among a plurality of biological elements, the sample comprising the plurality of biological elements,
the method being implemented by an electronic device, and comprising:
acquisition of an image of the sample;
preselection of at least one zone in the acquired image, from a database comprising a set of thumbnails of reference elements and a relevance index for each thumbnail of a reference element, the relevance index being increased, during the preselection, for each reference element thumbnail associated with a preselected zone;
display of each zone preselection in the image;
acquisition of the selection by an operator of at least one zone among the preselected zones, each selected zone corresponding to at least one element of interest; and
updating of the database according to the selected zone or zones acquired, at least one selected zone acquired being added as a thumbnail of a reference element in said base.

15. Method according to claim 14, wherein the preselection of at least one zone in the acquired image comprises:
the detection of a plurality of zones in the image, each zone corresponding to at least one biological element, and
the comparison of each zone detected with all the reference element thumbnails, the zone or zones being preselected according to said comparison.

16. Method according to claim 15, wherein, during the comparison, a value of at least one parameter relating to a corresponding biological element is calculated for each zone detected, and each parameter value calculated is compared with the corresponding parameter values of all the reference element thumbnails.

17. Method according to claim 16, wherein each parameter is chosen from the group consisting of: a shape, a diameter, a surface, a volume, a perimeter, a mass, a colour and a texture.

18. Method according to claim 14, wherein, during the preselection, a plurality of zones are preselected and, during the display, the zone preselections are displayed successively.

19. Method according to claim 18, wherein, during the display, the zone preselections are displayed in an order dependent on a degree of agreement of each preselection.

20. Method according to claim 15, wherein, during the display, the zone preselections are displayed in an order dependent on a degree of agreement of each preselection, the degree of agreement of each preselection being calculated according to the difference between the preselected zone and the reference element thumbnail associated during the comparison.

21. Method according to claim 14, wherein, during the preselection, the reference element thumbnails are taken into account in an order dependent on their respective relevance index.

22. Method according to claim 14, wherein each biological element is chosen from the group consisting of: a cytoplasm, an organite, a vesicle, a membrane, a nucleus, a cell, an intracellular pathogen, an extracellular pathogen, a unicellular organism, a pluricellular organism, a bacterium, a virus, a mycelium, a fragment of organism, an organism, an organ fragment, an organ, a tissue, a biological structure fragment, a biological structure, an anatomical structure fragment and an anatomical structure.

23. A non-transitory computer readable medium on which is stored a computer program containing software instructions which, when they are executed by a computer, implement a method comprising the following steps:
acquisition of an image of the sample;
preselection of at least one zone in the acquired image, from a database comprising a set of thumbnails of reference elements and a relevance index for each thumbnail of a reference element, and the relevance index being increased, during the preselection, for each reference element thumbnail associated with a preselected zone;
display of each zone preselection in the image;
acquisition of the selection by an operator of at least one zone among the preselected zones, each selected zone corresponding to at least one element of interest; and
updating of the database according to the selected zone or zones acquired, at least one selected zone acquired being added as a thumbnail of a reference element in said base.

24. Electronic device assisting the determination, in an image of a sample, of at least one element of interest among a plurality of biological elements, the sample comprising the plurality of biological elements, the electronic device comprising:
a first acquisition module configured to acquire an image of the sample;
a preselection module configured to preselect at least zone in the acquired image, from a database comprising a set of reference element thumbnails;
a display module configured to display each zone preselection in the image;
a second acquisition module configured to acquire the selection by an operator of at least one zone among the preselected zones, each selected zone corresponding to at least one element of interest; and
an updating module configured to update the database according to the selected zone acquired or zones, the updating module being configured to add, as a thumbnail of a reference element in said base, at least one selected zone acquired.
